(19) United States
(12) Patent Application Publication
(10) Pub. No.: US 2006/0292097 A1
(43) Pub. Date: Dec. 28, 2006

(54) Fragrance compositions comprising benzo[4,5]thieno{3,2-b]pyran-2-one

(76) Inventor: Luca Turin, London (GB)

Correspondence Address: MILES & STOCKBRIDGE PC, 1751 PINNACLE DRIVE, SUITE 500, MCLEAN, VA, 22102-3833, US

(21) Appl. No.: 10/552,459

(22) PCT Filed: Apr. 8, 2004
(86) PCT No.: PCT/US04/10829

§ 371 Date: Aug. 4, 2006

Related U.S. Application Data
(60) Provisional application No. 60/461,090, filed on Apr. 8, 2003.

(51) Int. Cl. A61K 8/49; C07D 495/02

(52) U.S. Cl. 424/65; 549/43; 510/102; 512/13

(57) ABSTRACT

The present case discloses compositions of benzo[4,5]thieno[3,2-b]pyran-2-one which are employed as aroma chemicals.

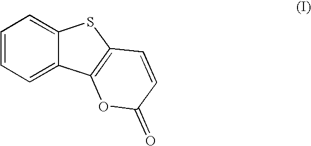

### FIELD OF THE INVENTION

The present invention relates generally to the field of flavorings and fragrances. More particularly, the present invention relates to perfumes and other fragrant articles based on aromachemicals which overcome the stability limitations and/or allergenic nature of the native compounds.

### BACKGROUND OF THE INVENTION

Many aromachemicals are used in the flavoring and fragrance industries. For example, citral has a lemon scent and as such is used as a flavor and/or fragrance in many articles of manufacture. However, many aromachemicals include isoprene units and/or benzene rings which are potentially susceptible to reaction and may result in a limited useful lifetime. Further, many essential oil fragrances have recently been determined to cause allergic reactions, and it is becoming increasingly difficult to bring such compounds to market.

Many aromachemicals, which are fundamental to the formation of various fragrances, have been placed on the allergens list and are being banned or restricted in many commercial regions. The bans or restrictions will undoubtedly have a considerable effect on the quality of various fragrances, largely because the reduction in the perfumers' palette makes the creation of certain notes virtually impossible.

It would be desirable to develop derivatives of aromachemicals that include benzene rings and/or isoprene units that do not similarly result in allergic reactions and/or which have improved useful lifetimes or other beneficial properties. Such additionally beneficial properties include improved odor intensity and stability. The present invention provides such fragrances and flavorings.

### SUMMARY OF THE INVENTION

Improved fragrances and flavorings that have improved physical and/or chemical properties relative to the parent compounds from which they are derived are disclosed. In particular, derivatives of aromachemicals that maintain or enhance the fragrance characteristics of the aromachemicals, while optionally lowering the allergenic properties, and which can optionally possess a longer shelf-life or other beneficial properties such as improved odor intensity compared to the parent compounds from which they are derived, are disclosed. Also disclosed are methods of making the derivatives, and articles of manufacture including the derivatives.

The parent compounds include one or more isoprene units and/or benzene rings, and the derivatives include a benzothiophene ring in place of at least one isoprene unit and/or benzene ring. The relative orientation of the benzothiophene ring can vary. The benzothiophene moiety can be unsubstituted, or substituted with one or two lower alkyl, preferably methyl groups. The alkyl groups and benzothiophene rings can optionally be substituted, for example, with electron donating groups, electron withdrawing groups, groups which increase the hydrophilicity or hydrophobicity, and the like. Where the benzothiophene includes one or more C₁₋₅ alkyl groups, these groups are preferably located in the 2 and/or 3-position.

Where these parent compounds further include one or more aldehyde groups, derivatives can be prepared in which at least one aldehyde group in the parent molecule is replaced with a nitrile, methyl ether or acetal group. The acetal groups can provide the compounds with a long lasting flavor or fragrance, where the acetals slowly hydrolyze to provide the parent aldehyde compounds. In some embodiments, suitable molecules include one or more aldehyde groups in addition to an isoprene unit and/or a benzene ring. In these embodiments, the aldehyde groups can also be derivatized as described herein.

Examples of suitable articles of manufacture include candles, air fresheners, perfumes, disinfectant compositions, hypochlorite (bleach) compositions, beverages such as beer and soda, denture cleanser tablets as described, for example, in U.S. Pat. No. 5,571,519, the contents of which is hereby incorporated herein by reference in its entirety, and flavored orally-delivered products such as lozenges, candies, and the like.

### DETAILED DESCRIPTION OF THE INVENTION

Improved fragrances and flavorings that exhibit improved physical and/or chemical properties compared to the parent compounds from which they are derived are disclosed. The present invention includes such improved fragrances and flavorings made by any method, regardless of whether the improved compound is literally “derived from” the parent compound by chemical modification of the latter compound. The improvements can be, for example, in the form of greater intensity and/or greater chemical stability without change in odor character. If greater intensity is desired, then the odorant structure is modified so as to increase the intensity of the odor, such as by increasing zinc-binding ability, without significantly changing odor character. If greater stability is desired, then one or more structural features responsible for chemical instability can be altered as described herein without significantly changing odor character.

I. Isodonic Molecules

The derivatives described herein are isodonic to the compounds from which they can be derived. By isodonic is meant “having essentially the same odor profile.” However, while the compounds may have essentially the same odor profile, they have improved stability, odor intensity and/or other improved physical and/or chemical properties.

The compounds from which the derivatives can be derived are aromachemicals, for example, specific odorant compounds present in essential oils. The derivatives can be prepared from the aromachemicals or the individual compounds, but need not be. That is, the compounds can be derived from synthetic strategies that do not involve using the aromachemicals, so long as the ultimate compound is a derivative of the oils or specific odorant compounds as described herein. All that is required is that the compounds are isodonic with the “parent” compounds.

Isodonic replacements, as defined herein, include replacement of isoprene moieties or benzene rings (phenyl groups) with benzothiophene rings. A benzothiophene can replace an isoprene or a benzene ring a) by connecting to the remainder of the molecule through any one of the free positions on its constituent atoms (vertices) or b) when the benzene ring in the parent compound is fused with another ring, the benzothiophene can substitute for it by being fused by one of its available bonds (sides).

In some embodiments, the parent compounds include an aldehyde group, nitrile group, methyl ether group and/or ester group in addition to the isoprene or phenyl groups. In these embodiments, in addition to the replacements described above, the following additional replacements can further be made: aldehyde-nitrile replacement, aldehyde-methyl ether replacement, aldehyde-acetal replacement, aldehyde-ester replacement, and inverses of these replacements (i.e., methyl ether-aldehyde and the like).

The odorant intensity and/or stability of an aromachemical can be improved by replacing a common chemical feature with another designed to alter the chemistry while leaving the basic structure, and therefore the odor itself, virtually untouched. Examples of suitable chemical features that can be replaced are described in more detail below.

Isoprene Unit/Benzothiophene Replacements and their Effect on Odor

Many aromachemicals include one or more isoprene units. The replacement of these units with a benzothiophene does not markedly change the odor character. However, the lone pair of electrons on the sulfur in the benzothiophene ring is believed to bind readily to Zn, which is believed to increase the odor intensity without significantly altering the odor type. One way to measure the odorant intensity is through zinc binding affinity.

Another advantage of replacing the isoprene unit (or, as discussed further below, a benzene ring) with a benzothiophene ring is that this produces a molecule with a higher molecular weight. The greater molecular weight can lower the volatility of the molecule, thereby potentially changing a top note to a middle note, or a middle note to a drydown note.

The procedure described herein for improving the performance of an odorant can be illustrated, for example, with citral. It is immediately applicable to any other odorants possessing the same structural features, namely an isoprene unit. Citral can be derivatized by replacing the isoprene unit with a benzothiophene ring. The benzothiophene ring can be substituted with one or two functional groups selected from the group consisting of halo, alkyl, preferably methyl, hydroxy, thiol, thioether, amine, carboxylic acid, ester, nitro, cyano, sulfonic acid, urea, and thiourea.

These simple procedures yield derivatives with odor profiles close to the aromachemicals or individual “parent” compounds themselves. Further, by replacing the isoprene units, the derivatives often have greater potency and far greater acid and bleach stability since the unstable feature, namely the double bond, has been removed. By replacing a benzene ring with a benzothiophene moiety, the potency is often increased.

The chemistry described herein generates a number of possible molecules from citral alone, all readily accessible by processes well known in the art. Examples of citral derivatives are shown below:

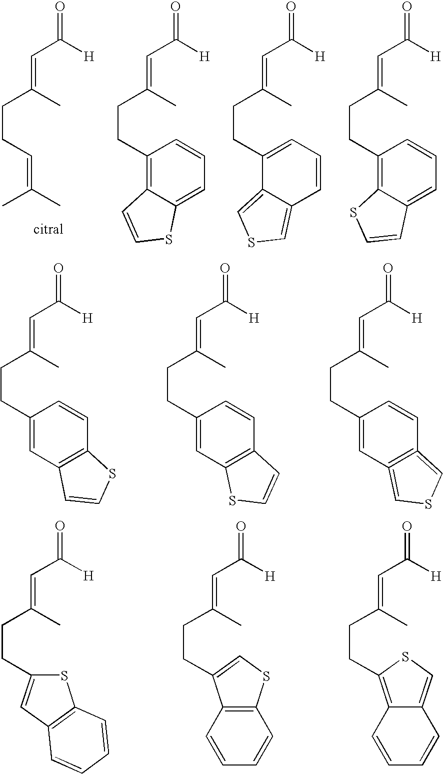

This method is immediately applicable to several other classes of odorants in order to increase their potencies. In each case the isoprene unit can be substituted with a benzothiophene to yield stronger odorants with similar odor profiles. Rose oxide is a floral; ionone, a woody violets; damascene, a fruity rose; sandanol, a sandalwood; limonene, a woody citrus; velvione, a musk; linalool, a floral-woody; and ethyl citronellyl oxalate, a musk.

Benzene/Benzothiophene Replacement

An additional odotopic replacement is a benzene ring for a benzothiophene ring. For example, when the phenyl rings in lilial, cyclamenaldehyde and bourgeonal are replaced with benzothiophene, not only do the vibrational spectra overlap and the novel derivatives have the same odor characteristics, but also the intensity of the odor is enhanced, compared to the parent compounds. Each of these compounds further includes an aldehyde group that can additionally be replaced with nitrile, methyl ether or acetal functionality. Synthetic methods for replacing a phenyl ring in a molecule with a benzothiophene molecule are well known to those of skill in the art.

Using the methods described herein, compounds like cinnamaldehyde, cyclamenaldehyde, lilial, and coumarin can be modified as shown below.

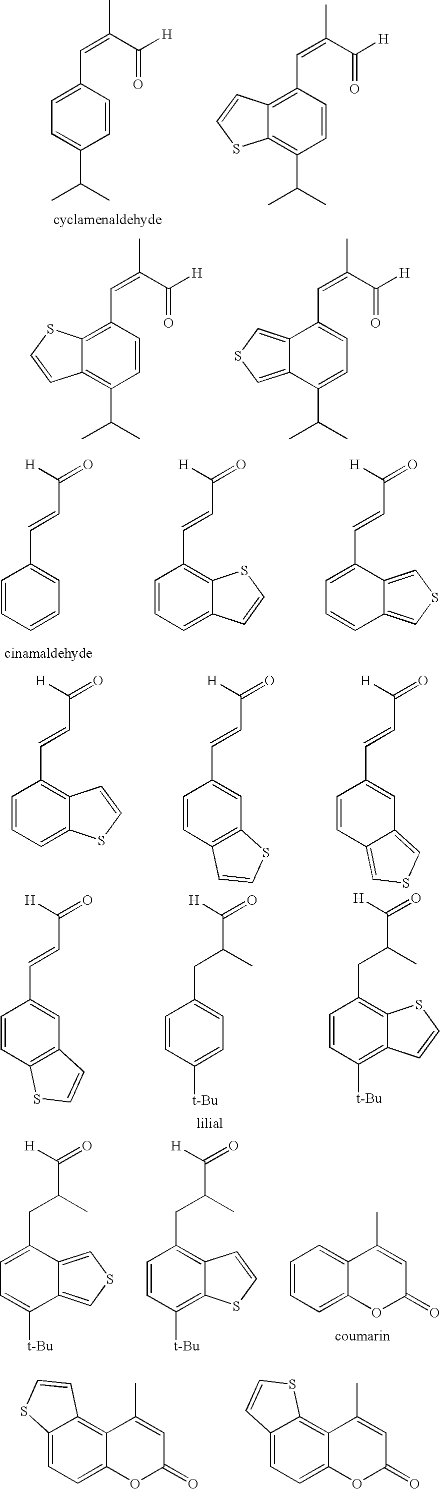

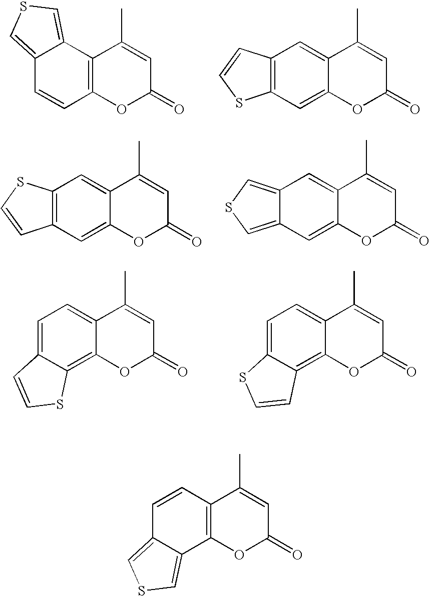

In addition to the replacements shown above, the benzothiophene rings can each be derivatized as described herein, for example, with alkyl groups at each of the various positions. Further, as discussed below, the aldehyde groups can also be subjected to odotopic replacement.

Aldehyde Replacement with Nitrile, Methyl Ester, or Acetal Groups

Many aromachemicals also include aldehyde groups. Odotopes of these aromachemicals can be prepared by replacing the aldehyde group with a nitrile, methyl ester or acetal group. The conversion of an aldehyde group to a nitrile group is well known in the art, and described, for example, in U.S. Pat. No. 5,892,092. The '092 patent teaches a process for forming nitriles from aldehydes. Acetal formation is well known to those of skill in the art, and generally involves reacting an aldehyde with an alcohol in the presence of an acid catalyst. The acetal is formed with loss of water. In use, when the acetal is present in an aqueous environment, the acetal can revert to the aldehyde, thereby providing a time-release form of the odorant. The aldehyde group in aromachemicals including an aldehyde in addition to one or more isoprene units or benzene rings can be converted to a methyl ether by reducing the aldehyde to a primary alcohol, and reacting the primary alcohol with a methylating agent such as methyl bromide or methyl iodide in the presence of a suitable base.

II. Aromachemicals that can be Modified Using the Chemistry Described Herein

The technology described herein has particular application to aromachemicals, in particular, aromachemicals that include lyral, and aldehydes and alcohols related to citral, geraniol, nerol and the like. Examples of aromachemicals that can be modified using the chemistry described herein are listed below:

Amyl cinnamyl (also known as 2-benzylidineheptanal and alpha-amyl cinnamic aldehyde)

Amyl cinnamyl alcohol (also known as 2-pentyl-3-phenylprop-2-ene-1-ol and alpha-amyl cinnamic alcohol)

cinnamyl alcohol (also known as cinnamic alcohol)

cinnamal (also known as 3-phenyl-2-propenal and cinnamic aldehyde)

citral (also known as 3,7-dimethyl-2,6-octadiene-1-al, mix of cis and trans isomers)

coumarin (also known as 1-benzopyran-2-one or cis-o-coumarinic acid lactone)

eugenol

geraniol

lyral (also known as hydroxymethyl-pentylcyclo-hexenecarboxaldehyde and 4,(4-hydroxy-4-methylpentyl)cyclohex-3-enecarbaldehyde

isoeugenol

benzoyl cinnamate (INCI), (also known as benzyl 3-phenyl-2-propenoate or cinnamein)

citronellol (also known as 3,7-dimethyl-6-octenol)

farnesol (also known as 3,7,11-trimethyldodeca-2,6,10-trienol

hexyl cinnamaldehyde (also known as alpha-hexyl cinnamaldehyde)

lilial (also known as lilestral, 2-(4-tert-butylbenzyl)proprionaldehyde, 4-(1,1-dimethylethyl)-alpha-methylbenzenepropanal, and p-tert-butyl-alpha-methylhydro cinnamaldehyde)

d-limonene (also known as (R)-p-mentha-1,8-diene

linalool,

damascones, and

gamma-methylionone ((also known as 3-methyl-4-(2,6,6-trimethyl-2-cyclohexen-1-yl)-3-butene-2-one.

Additionally, the compounds can be selected from anethole, anise oil, caraway oil, cardamom oil, carvone, coriander oil, eriodictyon, ethyl vanillin, fennel oil, glycyrrhiza, lavender oil, lemon oil, menthol, nutmeg oil, orange flower oil, peppermint, rosemary oil, rose oil, spearmint oil, thyme oil, tolu balsam and vanillin.

Additional examples include angelica, bergamotene, cyclolavandulal, citral, farnesal, ikema, isolauranal, phellandrene oxime and sorbinal oxime. In particular, citral oxime can be converted to geranonitrile.

Examples of parent ketones for these novel derivatives include alpha-ionone, beta-ionone, gamma-methyl ionone, irone alpha, methyl dihydrojasmonate, cis-jasmone, methyl amyl ketone, carvone, damascenone, alpha damascone, methyl beta-napthyl ketone, cassione, and menthone.

III. Articles of Manufacture Including the Aromachemical Derivatives

The derivatives described herein can be included in virtually any article of manufacture that can include the aromachemicals or other “parent compounds” from which they are derived. Examples include bleach, detergents, flavorings and fragrances, beverages, including alcoholic beverages, and the like. The derivatives can be used in applications like soaps, shampoos, body deodorants and antiperspirants, solid or liquid detergents for treating textiles, fabric softeners, detergent compositions and/or all-purpose cleaners for cleaning dishes or various surfaces, for both household and industrial use. Of course, the use of the compounds is not limited to the above-mentioned products, as they be used in other current uses in perfumery, namely the perfuming of soaps and shower gels, hygiene or hair-care products, as well as of body deodorants, air fresheners and cosmetic preparations, and even in fine perfumery, namely in perfumes and colognes. These uses are described in more detail below.

Perfume Compositions

The compounds can be used as perfuming ingredients, as single compounds or as mixture thereof, preferably at a range of at least about 30% by weight of the perfume composition, more preferably at a range of at least about 60% by weight of the composition. The compounds can even be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

In all cited applications, the derivatives can be used alone or in admixture with other perfuming ingredients, solvents or adjuvants of current use in the art. The nature and the variety of these co-ingredients do not require a more detailed description here, which, moreover, would not be exhaustive, and the person skilled in the art will be able to choose the latter through general knowledge and as a function of the nature of the product to be perfumed and of the desired olfactive effect.

These perfuming ingredients typically belong to chemical classes as varied as alcohols, aldehydes, ketones, esters, ethers, acetates, nitrites, terpene hydrocarbons, sulfur- and nitrogen-containing heterocyclic compounds, as well as aromachemicals of natural or synthetic origin. Many of these ingredients are described in reference textbooks such as the book of S. Arctander, Perfume and Flavor Chemicals, 1969, Montclair, N.J., USA, the contents of which are hereby incorporated by reference in their entirety, or in its more recent versions, or in other works of similar nature.

The proportions in which the derivatives can be incorporated in the various products vary within a large range of values. These values depend on the nature of the article or product that one desires to perfume and the odor effect searched for, as well as on the nature of the co-ingredients in a given composition when the compounds are used in admixture with perfuming co-ingredients, solvents or adjuvants of current use in the art.

As an example, the derivatives are typically present at concentrations between about 0.1 and about 10%, or even more, by weight of these compounds relative to the weight of the perfuming composition in which they are incorporated. Far lower concentrations than those mentioned above can be used when the compounds are directly applied for perfuming the various consumer products cited beforehand.

The compounds are relatively stable in typically aggressive media for perfumes. Accordingly, they can be used in detergents containing bleaching agents and activators such as, for example, tetraacetylethylenediamine (TAED), hypohalites, in particular hypochlorite, peroxygenated bleaching agents such as, for example, perborates, etc. The compounds can also be used in body deodorants and antiperspirants, for example, those containing aluminum salts. These embodiments are described in more detail below.

Conventional Detergent Ingredients

In addition to the derivatives described herein, the compositions herein include a detersive surfactant and optionally, one or more additional detergent ingredients, including materials for assisting or enhancing cleaning performance, treatment of the substrate to be cleaned, or to modify the aesthetics of the detergent composition (e.g., perfumes, colorants, dyes, etc.). The following are illustrative examples of detersive surfactants and other detergent ingredients.

Detersive Surfactants. Non-limiting examples of synthetic detersive surfactants useful herein, typically at levels from about 0.5% to about 90%, by weight, include the conventional C₁₁₋₁₈ alkyl benzene sulfonates (“LAS”) and primary, branch-chain and random C₁₀₋₂₀ alkyl sulfates (“AS”), the C₁₀₋₁₈ secondary (2,3) alkyl sulfates of the formula CH₃ (CH₂)ₓ(CH(CH₃)OSO₃⁻M⁺) and CH₃(CH₂)y(CH(CH₂CH₂)OSO₃⁻M⁺) wherein x and y are integers and wherein each of x and (y+1) is least about 7, preferably at least about 9, and M is a water-solubilizing cation, especially sodium, unsaturated sulfates such as oleyl sulfate, the C₁₀₋₁₈ alkyl alkoxy sulfates (“AEx S”; especially EO 1-7 ethoxy sulfates), C₁₀₋₁₈ alkyl alkoxy carboxylates (especially the EO 1-5 ethoxycarboxylates), the C₁₀₋₁₈ glycerol ethers, the C₁₀₋₁₈ alkyl polyglycosides and their corresponding sulfated polyglycosides, and C₁₂₋₁₈ alpha-sulfonated fatty acid esters. If desired, the conventional nonionic and amphoteric surfactants such as the C₁₂₋₁₈ alkyl ethoxylates (“AE”) including the so-called narrow peaked alkyl ethoxylates and C₆₋₁₂ alkyl phenol alkoxylates (especially ethoxylates and mixed ethoxy/propoxylates), C₁₂₋₁₈ betaines and sulfobetaines (“sultaines”), C₁₀₋₁₈ amine oxides, and the like, can also be included in the overall compositions. The C₁₀₋₁₈ N-alkyl polyhydroxy fatty acid amides can also be used. Typical examples include the C₁₂₋₁₈ N-methylglucamides. See WO 92/06154. Other sugar-derived surfactants include the N-alkoxy polyhydroxy fatty acid amides, such as C₁₀₋₁₈ N-(3-methoxypropyl)glucamide. The N-propyl through N-hexyl C₁₂₋₁₈ glucamides can be used for low sudsing. C₁₀₋₂₀ conventional soaps may also be used, however synthetic detergents are preferred. If high sudsing is desired, the branched-chain C₁₀₋₁₆ soaps may be used. Mixtures of anionic and nonionic surfactants are especially useful. Other conventional useful surfactants are listed in standard texts. See also U.S. Pat. No. 3,664,961 to Norris.

Preferred compositions incorporating only synthetic detergents have a detergent level of from about 0.5% to 50%. Compositions containing soap preferably comprise from about 10% to about 90% soap.

Although the detergent compositions herein can consist of only detersive surfactant and pro-fragrance, the said compositions preferably contain other ingredients commonly used in detergent products.

Builders

Detergent builders can optionally be included in the compositions herein to assist in controlling mineral hardness. Inorganic as well as organic builders can be used. Builders are typically used in fabric laundering compositions to assist in the removal of particulate soils.

The level of builder can vary widely depending upon the end use of the composition and its desired physical form. When present, the compositions will typically comprise at least about 1% builder. Liquid formulations typically comprise from about 5% to about 50%, more typically about 5% to about 30%, by weight, of detergent builder. Granular formulations typically comprise from about 10% to about 80%, more typically from about 15% to about 50% by weight, of the detergent builder. Lower or higher levels of builder, however, are not meant to be excluded.

Inorganic or detergent builders include, but are not limited to phosphate builders such as, the alkali metal, ammonium and allanolammonium salts of polyphosphates (exemplified by the tripolyphosphates, pyrophosphates, and glassy polymeric meta-phosphates), phosphonates, and phytic acid, and non-phosphorous builders such as silicates, carbonates (including bicarbonates and sesquicarbonates), sulphates, and aluminosilicates. Non-phosphate builders are required in some locales.

Organic builders suitable for use herein include polycarboxylate builders such as disclosed in U.S. Pat. No. 3,308,067 to Diehl; U.S. Pat. No. 4,144,226 to Crutchfield and U.S. Pat. No. 4,246,495 to Crutchfield, the contents of which are hereby incorporated herein by reference.

Soil Release Agents

Soil Release agents are desirably used in laundry detergents of the instant invention. Suitable soil release agents include those of U.S. Pat. No. 4,968,451 to Scheibel and Gosselink: Such ester oligomers can be prepared by (a) ethoxylating allyl alcohol, (b) reacting the product of (a) with dimethyl terephthalate (“DMT”) and 1,2-propylene glycol (“PG”) in a two-stage transesterification/oligomerization procedure and (c) reacting the product of (b) with sodium metabisulfite in water; the nonionic end-capped 1,2-propylene/polyoxyethylene terephthalate polyesters of U.S. Pat. No. 4,711,730 to Gosselink et al., for example those produced by transesterification/oligomerization of poly(ethyleneglycol)methyl ether, DMT, PG and poly(ethyleneglycol) (“PEG”); the partly- and fully-anionic-end-apped oligomeric esters of U.S. Pat. No. 4,721,580 to Gosselink, such as oligomers from ethylene glycol (“EG”), PG, DMT and Na-3,6-dioxa-8-hydroxyoctanesulfonate; the nonionic-capped block polyester oligomeric compounds of U.S. Pat. No. 4,702,857 to Gosselink, for example produced from DMT, Me-capped PEG and EG and/or PG, or a combination of DMT, EG and/or PG, Me-capped PEG and Na-dimethyl-5-sulfoisophthalate; and the anionic, especially sulfoaroyl, end-capped terephthalate esters of U.S. Pat. No. 4,877,896 to Maldonado, Gosselink et al., the latter being typical of SRA's useful in both laundry and fabric conditioning products, an example being an ester composition made from m-sulfobenzoic acid monosodium salt, PG and DMT optionally but preferably further comprising added PEG, e.g., PEG 3400. Another preferred soil release agent is a sulfonated end-capped type described in U.S. Pat. No. 5,415,807.

Other Optional Ingredients

The compositions herein can contain other ingredients such as enzymes, bleaches, fabric softening agents, dye transfer inhibitors, suds suppressors, and chelating agents, all well known within the art.

For purposes of defining detergent compositions of the present invention, the pH of the detergent composition is that which is measured at 1% concentration of the detergent composition in distilled-water at 20° C. The detergent compositions herein have a pH of from about 7.1 to about 13, more typically from about 7.5 to about 9.5 for liquid detergents and from about 8 to about 12 for granular detergents.

Formulation with Detergents with or without Conventional Perfumery Materials

While the derivatives described herein can be used alone and simply mixed with essential detergent ingredients, most notably surfactant, they can also be desirably combined into three-part formulations which combine (a) a non-fragranced detergent base comprising one or more synthetic detergents and (b) one or more of the derivatives described herein. In one embodiment, both aldehydes and acetals are present, such that the aldehydes provide desirable in-package and in-use (wash-time) fragrance, while the acetals provide a long-term fragrance to the laundered textile fabrics.

In formulating the present detergents, the fully-formulated fragrance can be prepared using numerous known odorant ingredients of natural or synthetic origin. The range of the natural raw substances can embrace not only readily-volatile, but also moderately-volatile and slightly-volatile components and that of the synthetics can include representatives from practically all classes of fragrant substances, as will be evident from the following illustrative compilation: natural products, such as tree moss absolute, basil oil, citrus fruit oils (such as bergamot oil, mandarin oil, etc.), mastix absolute, myrtle oil, palmarosa oil, patchouli oil, petitgrain oil Paraguay, wormwood oil, alcohols, such as farnesol, geraniol, linalool, nerol, phenylethyl alcohol, rhodinol, cinnamic alcohol, aldehydes, such as citral, Helional™, alpha-hexyl-cinnamaldehyde, hydroxycitronellal, Lilial™ (p-tert-butyl-alpha-methyldihydrocinnamaldehyde), methylaonylacetaldehyde, ketones, such as allylionone, alpha-ionone, beta-ionone, isoraldein (isomethyl-alpha-ionone), methylionone, esters, such as allyl phenoxyacetate, benzyl salicylate, cinnamyl propionate, citronellyl acetate, citronellyl ethoxolate, decyl acetate, dimethylbenzylcarbinyl acetate, dimethylbenzylcarbinyl butyrate, ethyl acetoacetate, ethyl acetylacetate, hexenyl isobutyrate, linalyl acetate, methyl dihydrojasmonate, styrallyl acetate, vetiveryl acetate, etc., lactones, such as gamma-undecalactone, various components often used in perfumery, such as musk ketone, indole, p-menthane-8-thiol-3-one, and methyl-eugenol. Likewise, any conventional fragrant acetal or ketal known in the art can be added to the present composition as an optional component of the conventionally formulated perfume (c). Such conventional fragrant acetals and ketals include the well-known methyl and ethyl acetals and ketals, as well as acetals or ketals based on benzaldehyde, those comprising phenylethyl moieties, or more recently developed specialties such as those described in a United States patent entitled “Acetals and Ketals of Oxo-Tetralins and Oxo-Indanes, see U.S. Pat. No. 5,084,440. Of course, other recent synthetic specialties can be included in the perfume compositions for fully-formulated detergents. These include the enol ethers of alkyl-substituted oxo-tetralins and oxo-indanes as described in U.S. Pat. No. 5,332,725; or Schiff Bases as described in U.S. Pat. No. 5,264,615. It is preferred that the pro-fragrant material be added separately from the conventional fragrances to the detergent compositions of the invention.

Formulation with Other Special-Purpose Fragrance Delivering Compounds

Detergents including the derivatives described herein may further, optionally, if desired, contain other known compounds having the capability to enhance substantivity of a fragrance. Such compounds include, but are not limited to, the aluminium alkoxides such as isobutylaluminium diferanylate as disclosed in U.S. Pat. No. 4,055,634; or the known titanate and zirconate esters or oligoesters of fragrant materials such as those disclosed in U.S. Pat. Nos. 3,947,574 and 3,779,932, the contents of each of which are hereby incorporated by reference. When using such organoaluminum, organotitanium or organozinc derivatives, they may be incorporated into the present formulations at their art-known levels.

Beverage Compositions

The improved flavorings described herein can be incorporated into beverages and impart various flavorings to the beverages. The preferred flavor is lemon, but additional flavors include rose, cinnamon, lime, and the like. The beverage composition can be a cola beverage composition, and can also be coffee, tea, dairy beverage, fruit juice drink, orange drink, lemon-lime drink, beer, malt beverages, or other flavored beverage. The beverages can be in liquid or powdered form.

The beverage compositions can also include one or more flavoring agents; artificial colorants; vitamin additives; preservatives; caffeine additives; water; acidulants; thickeners; buffering agents; emulsifiers; and or fruit juice concentrates.

Artificial colorants which may be used include caramel color, yellow 6 and yellow 5. Useful vitamin additives include vitamin B2, vitamin B6, vitamin B12, vitamin C (ascorbic acid), niacin, pantothenic acid, biotin and folic acid. Suitable preservatives include sodium or potassium benzoate. Salts which may be used include sodium, potassium and magnesium chloride. Exemplary emulsifiers are gum arabic and purity gum, and a useful thickener is pectin. Suitable acidulants include citric, phosphoric and malic acid, and potential buffering agents include sodium and potassium citrate.

In one embodiment, the beverage is a carbonated cola beverage. The pH is generally about 2.8 and the following ingredients can be used to make the syrup for these compositions: Flavor Concentrate, including one or more of the derivatives described herein (22.22 ml), 80% Phosphoric Acid (5.55 g), Citric Acid (0.267 g), Caffeine (1.24 g), artificial sweetener, sugar or corn syrup (to taste, depending on the actual sweetener) and Potassium Citrate (4.07 g). The beverage composition can be prepared, for example, by mixing the foregoing syrup with carbonated water in a proportion of 50 ml syrup to 250 ml of carbonated water.

In another embodiment, the beverage is a beer or malt beverage. Preferred flavorings for beer and malt beverages include lemon, lime and lemon-lime. Advantageously, the flavorings include citral derivatives in which one of both of the double bonds are replaced with a cyclopropane group, where the cyclopropane groups can, independently, be unsubstituted, or include one or two alkyl or substituted alkyl groups, preferably methyl groups. The amount of flavoring can be adjusted according to taste.

Orally-Delivered Products

Flavored food and pharmaceutical compositions including one or more of the derivatives described herein can also be prepared. The derivatives can be incorporated into conventional foodstuffs using techniques well known to those of skill in the art. Alternatively, the derivatives can be incorporated within polymeric particles, which can, in turn, be dispersed within and/or over a surface of an orally-deliverable matrix material, which is usually a solid or semi-solid substrate. When used in chewable compositions, the derivatives can be released into the orally-deliverable polymeric matrix material as the composition is chewed and held in the mouth, thus prolonging the flavor of the composition. In the case of dried powders and mixes, the flavor can be made available as the product is consumed or be released into the matrix material as the composition is further processed. When two flavors are combined with the polymeric particles, the relative amounts of the additives can be selected to provide simultaneous release and exhaustion of the compounds.

In one embodiment, the flavored composition includes an orally-deliverable matrix material; a plurality of water insoluble polymeric particles dispersed in the orally-deliverable matrix material, where the polymeric particles individually define networks of internal pores and are non-degradable in the digestive tract; and one or more derivatives as described herein entrapped within the internal pore networks. The derivatives are released as the matrix is chewed, dissolved in the mouth, or undergoes further processing selected from the group consisting of liquid addition, dry blending, stirring, mixing, heating, baking, and cooking. The orally-deliverable matrix material can be selected from the group consisting of gums, latex materials, crystallized sugars, amorphous sugars, fondants, nougats, jams, jellies, pastes, powders, dry blends, dehydrated food mixes, baked goods, batters, doughs, tablets, and lozenges.

Chewing Gum

A flavorless gum base can be combined with a citral or other suitable derivative as described herein to a desired flavor concentration. Typically, a blade mixer is heated to about 11° F., the gum base is preheated so that it is softened, and the gum base is then added to the mixer and allowed to mix for approximately 30 seconds. The flavored derivative is then added to the mixer and mixed for a suitable amount of time. The gum can be then removed from the mixer and rolled to stick thickness on waxed paper while warm.

Time Release Formulations

In one embodiment, the derivatives described herein are incorporated into a system which can release a fragrance in a controlled manner. These include substrates such as air fresheners, laundry detergents, fabric softeners, deodorants, lotions, and other household items. The fragrances are generally one or more derivatives of aromachemicals as described herein, each present in different quantities. U.S. Pat. No. 4,587,129, the contents of which are hereby incorporated by reference in their entirety, describes a method for preparing gel articles which contain up to 90% by weight of fragrance or perfume oils. The gels are prepared from a polymer having a hydroxy (lower alkoxy) 2-alkeneoate, a hydroxy (lower alkoxy) lower alkyl 2-alkeneoate, or a hydroxy poly (lower alkoxy) lower alkyl 2-alkeneoate and a polyethylenically unsaturated crosslinking agent. These materials have continuous slow release properties, i.e., they release the fragrance component continuously over a long period of time. Advantageously, all or a portion of those derivatives that include an aldehyde group can be modified to include an acetal group, which can cause the formulations to release fragrance over a period of time as the acetal hydrolyzes to form the aldehyde compound.

The present invention will be better understood with reference to the following non-limiting example.

### EXAMPLE 1

### Synthesis of benzo[4,5]thieno[3,2b]pyran-2-one

As shown below in Scheme 1, 2-mercaptobenzoic acid was reacted with trans-glutaconic acid in the presence of a catalytic amount of sulfuric acid to form benzo[4,5]thieno[3,2b]pyran-2-one. The chemistry is applicable to the synthesis of other compounds, where the mercapto-benzoic acid is further substituted with functional groups, such as alkyl groups, that do not interfere with the cyclization chemistry. Functional groups that otherwise might interfere with the cyclization chemistry can be present, provided they are protected. Protecting groups for such functional groups (i.e., hydroxy, amine, thiol and the like) are well known in the art and need not be discussed herein.

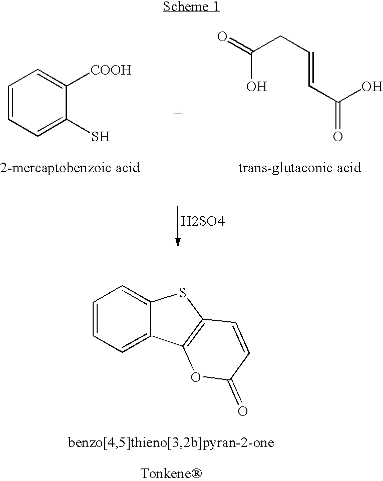

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. A derivative of an aromachemical comprising at least one isoprene unit or benzene ring, wherein at least one isoprene unit or benzene ring in the aromachemical is replaced with a benzothiophene ring, wherein the benzothiophene ring is optionally modified with one or more substituents selected from the group consisting of halo, alkyl, hydroxy, thiol, thioether, amine, carboxylic acid, ester, nitro, cyano, sulfonic acid, urea, and thiourea.

2. The derivative of claim 1, wherein the aromachemical comprises at least one isoprene unit.

3. The derivative of claim 1, wherein the aromachemical comprises at least one phenyl ring.

4. The derivative of claim 1, wherein the aromachemical comprises at least one aldehyde group, wherein further at least one aldehyde group in the aromachemical is replaced with a nitrite, methyl ether, ester or acetal group.

5. A composition comprising a compound of claim 1, together with at least on other perfuming ingredient, solvent, or adjuvant suitable for use in perfumery.

6. The composition of claim 5, wherein the compound is present in an amount that is at least 30 percent by weight of the composition.

7. The composition of claim 5, wherein the compound is present in an amount that is at least 60 percent by weight of the composition.

8. A perfuming composition or perfumed article containing as a perfuming ingredient a compound, or a mixture of compounds, of claim 1.

9. The perfuming composition of claim 8, wherein the compound or mixture of compounds is present in admixture with at least one other perfuming ingredient, solvent, or adjuvant suitable for use in perfumery.

10. A perfuming composition or perfumed article according to claim 8, in the form of a perfume or cologne, a soap, a bath or shower gel, a shampoo or other hair care product, a cosmetic preparation, a body deodorant or antiperspirant, an air freshener, a fabric detergent or softener or an all-purpose household cleaner.

11. A body deodorant or antiperspirant, containing as a perfuming ingredient a compound, or a mixture of compounds of claim 1.

12. The body deodorant or antiperspirant of claim 11, wherein the compound or mixture of compounds is present in admixture with at least one other perfuming ingredient, solvent, or adjuvant suitable for use in perfumery.

13. A detergent containing as a perfuming ingredient a compound, or a mixture of compounds of claim 1.

14. The detergent of claim 13, wherein the compound or mixture of compounds is present in admixture with at least one other perfuming ingredient, solvent, or adjuvant of current use in the art.

15. A bleach composition comprising an aromachemical derivative according to claim 1.

16. A beverage comprising an aromachemical derivative according to claim 1

17. The beverage of claim 16, wherein the beverage is selected from the group consisting of beer, malt liquor, lemonade and cola.

18. A flavored orally-delivered product comprising an aromachemical derivative according to claim 1.

19. A method to improve, enhance. or modify the odor of a perfuming composition or a perfumed article comprising adding to said composition or said article an effective amount of a compound or a mixture of compounds of claim 1.

20. The method of claim 19, wherein the compound or mixture of compounds is present in admixture with at least one other perfuming ingredient, solvent, or adjuvant suitable for use in a flavoring and/or fragrance.

21. A composition comprising benzo[4,5]thieno[3,2b]pyran-2-one, together with at least on other perfuming ingredient, solvent, or adjuvant suitable for use in perfumery.

22. The composition of claim 21, wherein the benzo[4,5]thieno[3,2b]pyran-2-one is present in an amount that is at least 30 percent by weight of the composition.

23. The composition of claim 21, wherein the benzo[4,5]thieno[3,2b]pyran-2-one is present in an amount that is at least 60 percent by weight of the composition.

24. A perfuming composition or perfumed article comprising benzo[4,5]thieno[3,2b]pyran-2-one as a perfuming ingredient a compound.

25. The perfuming composition of claim 24, wherein the benzo[4,5]thieno[3,2b]pyran-2-one is present in admixture with at least one other perfuming ingredient, solvent, or adjuvant suitable for use in perfumery.

26. A perfuming composition or perfumed article according to claim 24, in the form of a perfume or cologne, a soap, a bath or shower gel, a shampoo or other hair care product, a cosmetic preparation, a body deodorant or antiperspirant, an air freshener, a fabric detergent or softener or an all-purpose household cleaner.

27. A body deodorant or antiperspirant, comprising benzo[4,5]thieno[3,2b]pyran-2one as a perfuming ingredient.

28. The body deodorant or antiperspirant of claim 27, wherein the benzo[4,5]thieno[3,2b]pyran-2-one is present in admixture with at least one other perfuming ingredient, solvent, or adjuvant suitable for use in perfumery.
